(19) United States
(12) Patent Application Publication
(10) Pub. No.: US 2019/0053728 A1
(43) Pub. Date: Feb. 21, 2019

(54) System and method for activation recovery interval imaging of cardiac disorders

(71) Applicant: Regents of the University of Minnesota, Minneapolis, MN (US)

(72) Inventors: Ting Yang, Minneapolis, MN (US); Bin He, Minneapolis, MN (US)

(21) Appl. No.: 15/998,821

(22) Filed: Aug. 16, 2018

Related U.S. Application Data
(60) Provisional application No. 62/546,329, filed on Aug. 16, 2017.

(51) Int. Cl. A61B 5/04; A61B 5/0472; A61B 5/0408; A61B 5/00; A61B 5/0468

(52) U.S. Cl. CPC A61B 5/04012; A61B 5/0472; A61B 5/0464; A61B 5/0044; A61B 5/0468; A61B 5/0408

(57) ABSTRACT

A system and method for 3D activation recovery interval (ARI) imaging is provided that may be used to determine the spatial pattern of cardiac activation and recovery by reconstructing ARI maps noninvasively. This may be used for patients suffering from premature ventricular contraction (PVC), for example. Recovery plays an important role in the susceptibility to cardiac arrhythmias. Given the inhomogeneous and dynamic nature of the recovery pattern, a 3D ARI imaging can provide important information guiding determination of the underlying arrhythmogenesis. Additionally, 3D ARI imaging has the advantage of being able to be extracted from multiple, simultaneously recorded electrograms and reflects the dynamics and spatial characteristics of repolarization.

### CROSS-REFERENCE TO RELATED APPLICATIONS

This application claims the benefit of U.S. Provisional Patent Application Ser. No. 62/546,329 filed on Aug. 16, 2017 and entitled “System and Method for Activation Recovery Interval Imaging of Cardiac Disorders.”

### STATEMENT REGARDING FEDERALLY SPONSORED RESEARCH

This invention was made with government support under HL080093 awarded by the National Institutes of Health and CBET-0756331 awarded by the National Science Foundation. The government has certain rights in the invention.

### BACKGROUND

Dispersion of ventricular repolarization due to abnormal activation contributes to the susceptibility to cardiac arrhythmias. It has been shown that calcium plays an important role in cardiac excitation-contraction coupling and calcium leak is determinant in pathological conditions such as heart failure. Studies have shown the relationship between inhomogeneity of ventricular repolarization and enhanced ventricular arrhythmia vulnerability.

### SUMMARY OF THE DISCLOSURE

Studies of refractory period, transmembrane action potential duration, and activation recovery interval (ARI) have been used to understand the properties of ventricular repolarization. Previously, it has been reported to estimate activation and repolarization times from unipolar electrograms. By comparing the difference between the repolarization time and the activation time with simultaneously recorded in vivo transmembrane action potential durations, this method has been shown to be a good estimation of action potential durations (APDs). Later, ARI was defined as the interval between times of activation and repolarization, and was used in two animal studies which demonstrated high correlation between ARI from unipolar electrograms and refractory periods or action potential durations under a variety of conditions. In such a method, activation and repolarization time is defined as the time of minimum derivative of the QRS and the time of maximum derivative of the T wave in unipolar electrograms, respectively.

T wave polarities correspond to different repolarized regions, and there has been a controversy over the determination of repolarization time for positive T waves in unipolar electrograms. A previous work proposed an alternative method in which the minimum derivative on the downslope of the positive T waves was chosen to represent the local repolarization. This study and other following studies have observed a better correlation between ARI and MAP90% (90% repolarization of monophasic action potential recordings). However, the theoretical rationale behind this alternative method remains unclear. To justify the use of such a method, others have validated through a simple theoretical model that the maximum derivative of T waves should always be used regardless of T wave polarities. And finally, a more recent study identified the sources of measurement bias and supported previous assertions that an alternative method does not reliably represent either repolarization time or MAP90%.

Besides validating the use of ARI from invasive recordings, it is desirable to discover parameters that could reflect repolarization properties from noninvasive recordings. It has been shown that indices derived from 12-lead ECG are inadequate and even high-resolution body surface potential map (BSPM) has its limits in identifying local changes in repolarization. In this case, cardiac electrical imaging technique comes as a better choice in the sense of being noninvasive and time-saving and providing detailed cardiac activities. Based on the relationship between epicardial potential and body surface potential distribution, epicardial potential imaging such as noninvasive electrocardiographic imaging (ECGI) was developed to reconstruct epicardial potentials from BSPMs and has been applied to a variety of conditions. Regularization techniques on solving the epicardial potential have been proposed and compared among the existing techniques. Activation time imaging on both the epicardial and endocardial surface was also proposed and have been successfully applied to human subjects. Most recently, a quantitative comparison was made between ECGI and invasive epicardial electrography in four normal anesthetized dogs for the purpose of reconstructing epicardial activation and recovery patterns. However, since ventricular repolarization is inhomogeneous, one can only infer deep source activities from epicardial potential 2D maps.

In three-dimensional (3D) space, the moving dipole model was first used to represent the cardiac activity, and it was applied to localize the origins of ventricular activation. Later, distributed source models were proposed and have gained much attention since cardiac electrical activity is distributed throughout the heart. A 3D cardiac electrical imaging (3DCEI) technique was developed to estimate the equivalent current density at each cardiac source location from BSPMs and derived the activation time from the time course of current density. This method has been quantitatively evaluated in animal models under different conditions such as pacing, ventricular tachycardia, drug-induced QT prolongation and non-ischemic heart failure. 3DCEI was also applied to atrial arrhythmias by extracting frequency features from the reconstructed current density. Several other efforts were made to reconstruct transmembrane potentials in 3D space noninvasively. These methods have been tested under cardiac disease conditions, such as heart failure and myocardial infarction. Most recently, a machine learning algorithm was incorporated to localize the onset activation location in a premature ventricular contraction (PVC) patient and five cardiac resynchronization therapy (CRT) patients.

Premature ventricular contraction (PVC) is a type of ectopic beat caused by an ectopic pacemaker in the ventricles. It is one of the common arrhythmias and its prevalence varies depending on many factors, such as age, sex, race, and the presence of heart disease. In a large cross-sectional analysis of the 15,792 individuals (aged 45-65 years) from the U.S., the prevalence of PVC is >6% among these adults based on a 2-minute ECG. Pacing is one way to study the cardiac activities during ectopic beats. Studies have shown that ventricular repolarization potential distributions during ectopic beats were influenced by transventricular gradients (gradients from one side of the heart to the other) and the longest ARIs were located at the sites of earliest activation and shortest at the latest activation areas during pacing.

The present disclosure addresses the aforementioned drawbacks by providing a system and method for determining the recovery properties of ventricles by reconstructing ARI distributions from the time course of current density. Previously, ventricular recovery patterns could only be reflected from epicardial potentials noninvasively or from endocardial potentials invasively. Although the activation pattern may remain unchanged, recovery could vary and thus plays an important role in the susceptibility to cardiac arrhythmias. Given the inhomogeneous and dynamic nature of the recovery pattern, a 3D ARI imaging can provide important information guiding determination of the underlying arrhythmogenesis. The 3D ARI imaging technique may be evaluated by comparing the imaged ARI maps with those extracted from CARTO systems.

In one embodiment the invention provides a method for cardiac activation—repolarization imaging. The method includes steps of: acquiring image data including a heart of a subject; generating a three-dimensional representation of the subject based on the image data and a location of at least one sensor associated with the subject; acquiring electrophysiological data using the at least one sensor; reconstructing an activation recovery interval image of the heart of the subject based on the electrophysiological data and the three-dimensional representation of the subject; and providing the activation recovery interval image of the heart to a user.

In another embodiment, the invention provides a system for cardiac activation—repolarization imaging. The system includes: an imaging system for acquiring image data of a heart of a subject; at least one sensor associated with the subject for acquiring electrophysiological data; and a processor. The processor is to: generate a three-dimensional representation of the subject based on the image data and a location of the at least one sensor, reconstruct an activation recovery interval image of the heart of the subject based on the electrophysiological data and the three-dimensional representation of the subject, and provide the activation recovery interval image of the heart to a user.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1A is a flowchart depicting one configuration of the present disclosure.

FIG. 1B is another flowchart depicting one configuration of the present disclosure.

FIG. 2 is a graph of a unipolar electrogram, its derivative, and an example ECG trace that may be used to generate a form of ARI data.

FIG. 3A is a depiction of an ARI image analysis that may be displayed for a user.

FIG. 3B is another depiction of an ARI image analysis that may be displayed for a user.

FIG. 4 is a boxplot comparing ARI image distributions to extracted ARI distributions from a CARTO unipolar electrogram.

FIG. 5 is a scatter plot of the results of an example comparison following the process of FIG. 1B.

FIG. 6 is a schematic for one configuration of the present disclosure.

FIG. 7 is another schematic for one configuration of the present disclosure.

### DETAILED DESCRIPTION

Three-dimensional ARI imaging may be used to determine the spatial pattern of cardiac activation and recovery with the ability to reconstruct ARI maps noninvasively. This may be used for patients suffering from premature ventricular contraction (PVC), for example. Quantitative comparisons may be made between imaging results and clinical recordings. Previously, ventricular recovery patterns could only be reflected from epicardial potentials noninvasively or from endocardial potentials invasively. Although the activation pattern may remain unchanged, recovery could vary and thus plays an important role in the susceptibility to cardiac arrhythmias. Given the inhomogeneous and dynamic nature of the recovery pattern, a 3D ARI imaging can provide important information guiding determination of the underlying arrhythmogenesis. Additionally, 3D ARI imaging has the advantage of being able to be extracted from multiple, simultaneously recorded electrograms and reflects the dynamics and spatial characteristics of repolarization.

Systems and methods are provided for a three-dimensional (3D) approach to imaging activation and repolarization in the heart. The activation and repolarization interval of cardiac processes are imaged quantitatively. Advantageously, imaging can be achieved over the 3D myocardium using electrical recordings acquired noninvasively from the surface of the body. Furthermore, such imaging can be accomplished from electrical recordings obtained from a catheter, or from magnetic recordings outside of the body.

In some configurations, 3D cardiac electric imaging methodology is applied to activation and repolarization interval (ARI). ARI imaging is extended to 3D cardiac volume. The results of 3D ARI imaging are useful for, for example, for diagnoses, as they are highly correlated with ventricular arrhythmia such as Premature Ventricular Contraction (PVC). The disclosed 3D ARI imaging can help determine the spatial pattern of activation and repolarization and, in certain implementations, to construct, noninvasively, ARI maps in patients with PVC or other cardiac disorders.

In other configurations, rather than mapping PVC, the disclosed approach can be applied to other cardiac disorders, such as bundle branch block, early repolarization, cardiac memory, Wolff-Parkinson-White syndrome and ischemia. In various configurations, potential applications include guiding ablation treatment of cardiac arrhythmias.

3D ARI imaging is superior to pure activation imaging in the 3D heart. It offers enhancements over conventional two-dimensional (2D) cardiac surface approaches.

In one configuration, 3D ARI is performed from imaging from recordings made from an array of electrodes of a catheter, including simultaneously or sequentially recorded electrograms by electrodes of a catheter. The relationship of catheter recordings and cardiac activity can be established by solving Poisson's equation. The activation repolarization interval at each point within 3D cardiac volume can then be determined.

Another configuration is to use combined body surface electrical recordings and catheter electrical recordings to image activation repolarization interval at each point within myocardial volume.

In one configuration, combined electrical and magnetic recordings may be used to image the activation repolarization interval at each point within the 3D heart, or to use magnetic recordings, such as magnetocardiographic measurements, for the ARI imaging over the 3D heart. The relationship between magnetic recordings and cardiac electrical activity can be established by solving Poisson's equation with regard to the magnetic field. The ARI imaging principles as described herein can then be applied to the relationship to estimate ARI from magnetic recordings.

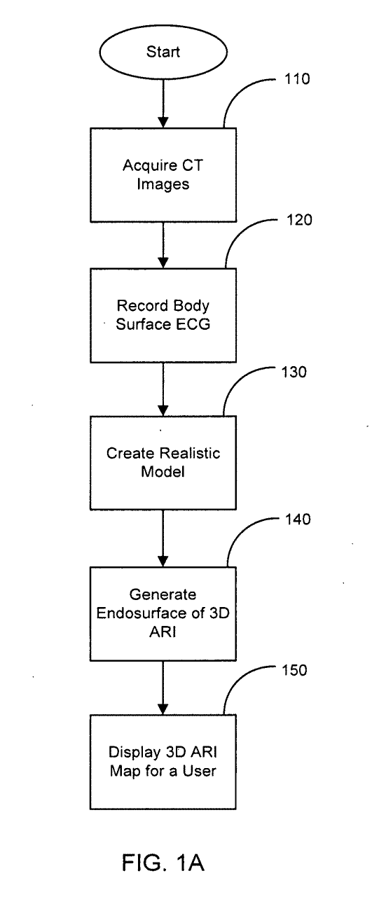

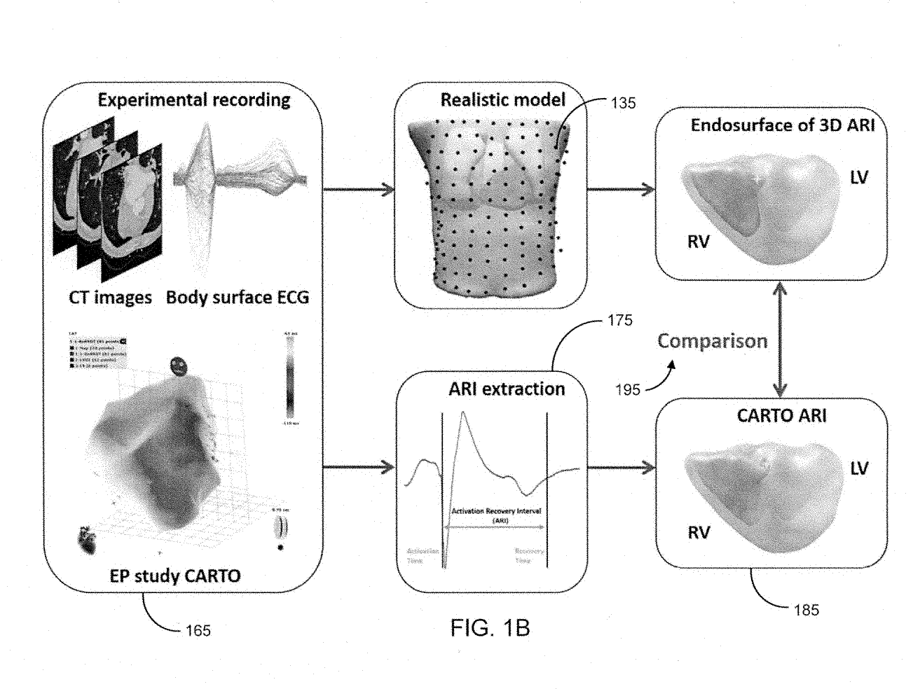

Referring to FIG. 1A, in one configuration CT images may be collected at step 110 prior to an electrophysiology (EP) study and separately from body surface recordings obtained at step 120 for a body surface potential map (BSPM). The CT images may be registered based on important cardiac anatomical landmarks, such as the apex or the septum between two ventricles. The CT images may include cross-sectional CT slices showing detailed heart geometry and torso boundaries. The recordings at step 120 may include multiple-channel ECGs and local activation time maps recorded during an EP study. Registration errors may be minimized with the assistance of an appropriate registration software system, such as the Curry 6.0 system. A realistic model including torso, lungs, epicardium, ventricles, and the like may be built at step 130 from the CT images by using a similarly appropriate commercial software for segmentation and registration. The locations of the surface electrodes may be reflected on this model. An endocardial surface of ARI maps may be generated at step 140 by applying ARI imaging. These 3D ARI maps may then be displayed for a user at step 150 to be used in diagnosing a subject.

Referring to FIG. 1B, in one configuration CT images may be collected at the same time as ECG recordings and the generation of an EP CARTO map at step 165. Electrodes 135 used in the surface map, such as the ECG electrodes, may have their locations mapped onto the realistic model. An ARI may be extracted from the CARTO EP map at step 175 and a CARTO ARI map may be created at step 185 using the extracted ARI from step 175. This CARTO ARI map may be compared at step 195 to the Endosurface 3D ARI map created according to FIG. 1A.

In some configurations, body surface recording may be performed prior to the EP study, such as one day before the EP study. Electrodes may be used for the body surface recordings, such as Ag—AgCl carbon electrodes, and may be placed in appropriate locations on the subject. Appropriate locations may include on the front and back of patients when they are in a Fowler's position. A different number of electrodes may be placed on the front than are placed on the back. The locations of the electrodes may be digitized by using an electromagnetic digitizer, such as a Fastrak digitizer. In some configurations, the locations of anatomical landmarks on the body surface may also be digitized to aid co-registration of body surface electrodes with the CT images. These landmarks may include the acromion process, jugular notch, xiphoid process, umbilicus, and the like. Thus, the locations of the electrodes may be mapped onto a three-dimensional representation of the subject's body, particularly the subject's torso region, and the electrical activity of the heart can be determined based on the EP data obtained from the electrodes in relation to the subject's anatomical information, for example using the calculations described below. The electrodes may then be connected to a recording system having a sufficiently high sampling frequency, such as a BioSemi ActiveTwo system with a sampling frequency of 2 kHz, to record body surface ECGs; in various configurations, sampling frequencies of between 500 Hz-5 kHz may be used. Motion artifacts may be mitigated during recording using any appropriate method, such as by slowing the subject's respiration.

In one configuration, CARTO files may be collected after each EP study, which may include endocardial surfaces of either or both ventricles, endocardial local activation time maps, ablation sites or unipolar electrograms, and the like. If unipolar electrograms are used, they may be obtained sequentially, site-by-site by the CARTO system. The endocardial surfaces of ventricles may be registered with the segmented heart from the CT images using, for example, a four degrees-of-freedom (locations and scale) rigid body registration method. The CARTO endocardial geometries may be automatically aligned based on similarities in shape without rotation. Registration may be performed by minimizing the spatial distances between the CARTO surfaces and the endocardial surfaces from the CT images. The average spatial distance between CARTO surfaces and endocardial CT surfaces, namely the average registration error, may be recorded along with the electrode data. In one configuration, after registering body surface electrodes with the realistic model, a boundary element model (BEM) comprising torso, lungs and heart may be built, and the conductivities may be recorded.

By implementing an ARI imaging technique as described below, a 3D ARI map may be reconstructed. Alternatively, ARI may be extracted from unipolar electrograms and interpolated to create a CARTO ARI map. A 3D ARI map may then be compared with the CARTO ARI map quantitatively.

In one configuration, modeling used for ARI imaging may be based on bi-domain theory. The discrete cellular architecture may then be represented by a macroscopic continuum model which may include two domains: the intracellular and extracellular domain. These two domains may be connected by transmembrane currents flowing across a theoretical membrane with no thickness. Assuming a quasi-static condition, the electrical field within the volume conductor is governed by an adaptation of the Poisson equation:

∇·[(Gᵢ+Gₑ)∇ϕₑ]=∇·\vec{j}_{eq} (1)

where Gᵢ and Gₑ are intracellular and extracellular conductivity tensors respectively, ϕₑ is the extracellular potential and ϕₘ is the transmembrane potential. If we define equivalent current density to be \vec{j}_{eq}=−Gᵢ∇ϕₘ, then the above equation can be rewritten as:

∇·[(Gᵢ+Gₑ)∇ϕₑ]=∇·\vec{j}_{eq} (2)

Thus, \vec{j}_{eq} can be the equivalent cardiac sources for generating cardiac electrical activities. To model the distributed current sources, the whole ventricular myocardium may be discretized into N grid points. An orthogonal triple dipole may be placed at each grid point to represent a local vector field with an arbitrary direction.

According to the analytical derivation, at any time the electrical potential at a given point on the torso surface is a linear superposition of all the potential fields generated by cardiac sources at that time. In one configuration, by applying BEM theory after the tessellation of relevant surfaces (torso, lungs and epicardium), this linear relationship may be reflected in the discrete matrix equation:

ϕ_{b}(t)=LJ(t) (3)

where ϕ_{b}(t) is a M×1 vector of body surface potentials at M electrode positions at time t and J(t) is a 3N×1 vector of the equivalent current density at N source locations at time t. L is an M×3N transfer matrix with the lead field between one electrode and one cardiac source is a 1×3 vector.

In some configurations, 3N is much larger than M. Finding J(t) given ϕ_{b}(t) is an ill-posed inverse problem, which needs mathematical regularization. A generalized Tikhonov regularization may be applied to solve this inverse problem:

$${min}_{J ( t )} ( {‖ {φ}_{b} ( T ) - LJ ( t ) ‖}_{2}^{2} + λ {‖ WJ ( t ) ‖}_{2}^{2} ) \qquad ( 4 )$$

Where W is the depth weighting matrix calculated from L and Δ is the regularization parameter determined by an L-curve method.

After solving the inverse problem on QRS complexes and T waves separately, the time course of the equivalent current density at each source location may be obtained. By definition, the amplitude of equivalent current density is proportional to the spatial gradient of transmembrane potential. During the process of activation (or recovery), the spatial distribution of equivalent current density is dominated by its values at the interface between the activated and non-activated myocardium (or between the recovered and non-recovered myocardium). According to the peak criteria, the activation time τₐ (or recovery time τᵣ) is when the amplitude of J(t) reaches its maximum during the duration time T at a fixed location P:

$$τ ( p ) = {max}_{t ∈ T} ( J ( p , t ) ) \qquad ( 5 )$$

The activation recovery interval (ARI) is defined as the interval between recovery and activation, so ARI at location P is: ARI (p)=τᵣ(p)−τₐ(p). Since the ARI value is the difference between the recovery time and the activation time, it is a relative value. In some configurations, ARI imaging may reconstruct the spatial pattern of ARI instead of obtaining the absolute value of ARI. Therefore, ARI imaging results may be further linearly scaled based on the range of ARI estimated from BSPM. The longest possible ARI value corresponds to the length of a PVC beat and the shortest possible ARI value the sum of QRS length plus ST length.

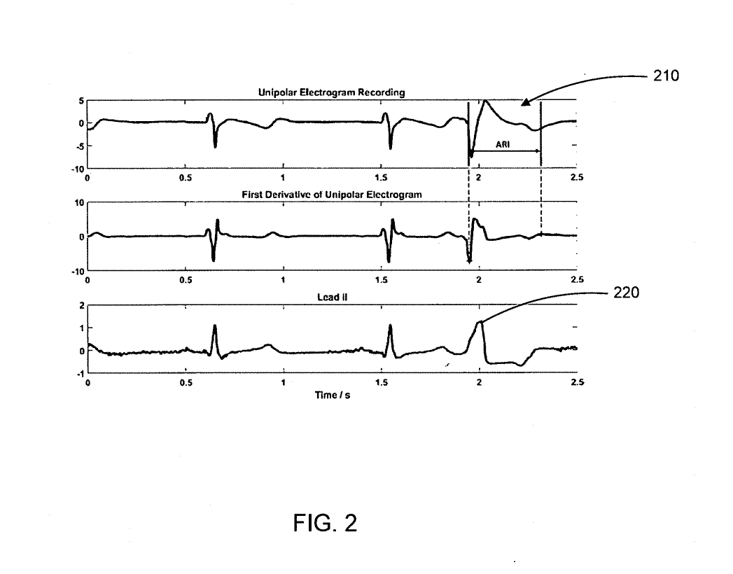

Referring to FIG. 2, in one configuration, ARI values 210 may be extracted from unipolar electrograms. Unipolar electrograms may be included in CARTO files. Any appropriate electrogram recording device may be used for recording electrogram data, such as a quad mapping catheter. In one example, at each recording site, four unipolar electrograms (M1 to M4) may be recorded along with 12-lead ECGs by CARTO system. The ECG data may be used to identify the PVC beats 220. An electrogram signal and the first derivative of this signal may be plotted along with a selected lead from the ECGs to help identify PVC beats 220. The time length of CARTO recording may be 2.5 seconds in all the channels. The minimum derivative in QRS and the maximum derivative in T wave may be automatically detected by an appropriate algorithm and marked as activation time and recovery time respectively. Then, within the time segment of QRS complexes (or T wave), the minimum derivative (or maximum derivative) may be automatically determined. The time window of calculating the first derivative is within a certain number of samples dependent upon the sampling frequency, such as 20 samples with a sampling frequency of 1 kHz, which means the first derivative at time point i is the difference between the magnitude at time point i+10 and i−10.

A window length may be selected to reduce the influence of high frequency noise. Therefore, the accuracy of determining the minimum (or maximum) derivative may be on the order of milliseconds. However, this accuracy does not affect the accuracy of determining ARI from unipolar electrograms because ARI is a difference between recovery time and activation time. What influence such an auto-detection algorithm may have on determining the time point with the minimum derivative may be imposed in the same way as determining the time point with the maximum derivative. The results of ARI extraction from CARTO-recorded unipolar electrograms may be considered as a ground truth for verification of ARI imaging. ARIs at multiple recording sites may be interpolated to the CARTO endocardial surface to get an endocardial ARI map for each patient by an inverse distance weighted (IDW) interpolation method.

In one example, the first derivative of the unipolar electrogram (M1 channel) may be calculated. In this example, the M1 signal and the first derivative of this signal along with Lead II from 12-lead ECGs were used to help identify PVC beats. Then, within the time segment of QRS complexes (or T wave), the minimum derivative (or maximum derivative) was automatically determined. The time window of calculating the first derivative is 20 samples with a sampling frequency of 1 kHz which means the first derivative at time point i is the difference between the magnitude at time point i+10 and i−10. Window length of 20 samples was to reduce the influence of high frequency noise. Therefore, the accuracy of determining the minimum (or maximum) derivative is 20 ms.

In some configurations, to evaluate the performance of ARI imaging, a quantitative comparison may be made between two endocardial ARI maps: one extracted from the 3D ARI map and the other being an interpolated CARTO ARI map. Each subject may have one interpolated CARTO ARI map and a number of 3D ARI maps, such as 10 for example, reconstructed from an equivalent number of PVC beats in BSPM for comparison. For these comparisons, unipolar recording sites may be spread throughout the whole chamber of either left ventricle or right ventricle, which enables extracting the endocardial ARI map from the 3D ARI map and to project the endocardial CARTO ARI map to the endocardial surface from the CT images. Projection from one volume to another may be done by finding the closest spatial point in one volume corresponding to the point in the second volume. Therefore, comparison may be made on the endocardial surface from the CT images.

In some configurations, because of the small number of unipolar recording sites available for a particular subject and the accompanying small available region of the CARTO endocardial map, a 3D ARI map may be projected onto and CARTO ARIs may be interpolated onto the same CARTO endocardial surface for comparison. Statistical analysis may be performed, including determining a correlation coefficient (CC) and relative error (RE) between the two endocardial ARI maps, localization error between the longest ARI sites (centers of mass with longest ARIs) in the two maps (LE-map), and spatial distance between the earliest activated site and the longest ARI site in the 3D ARI map (Dis-actiari). The standard deviation of CC is the standard deviation among ten obtained CCs, not of the distribution of any individual CC. CC, RE, LE-map and Dis-actiari may be calculated as following:

$$\begin{matrix}cc = \frac{{\sum }_{i} ( {IARI}_{i} - \overline{IARI} ) ( {MARI}_{i} - \overline{MARI} )}{\sqrt{{\sum }_{i} {( {IARI}_{i} - \overline{IARI} )}^{2}} \sqrt{{\sum }_{i} {( {MARI}_{i} - \overline{MARI} )}^{2}}} & ( 6 ) \\ RE = \sqrt{\frac{{\sum }_{i} {( {IARI}_{i} - {MARI}_{i} )}^{2}}{{\sum }_{i} {MARI}_{i }^{2}}} & ( 7 ) \\ LE - map = ‖ {S}_{l} - {S}_{M} ‖ & ( 8 ) \\ Dis - actiari = ‖ {S}_{acti} - {S}_{ari} ‖ & ( 9 )\end{matrix}$$

where IARIᵢ and MARIᵢ represent the imaged and measured ARI at iₜₕ location, IARI and MARI are the average values of the two sequences; S_{I} and S_{M} are the spatial locations of imaged and measured longest ARI sites respectively, S_{acti} is the earliest activated site in the activation time map, and Sₐᵣᵢ is the longest ARI site in the 3D ARI map.

In an example configuration, BSPMs were recorded in 10 PVC patients (Male=4, Female=6, average age=47.8±11.7 years old) before the electrophysiology (EP) procedure with spontaneous PVCs detected. None of the patients had undergone an ablation procedure previously. The number of PVCs was counted by Holter monitors. CT images were collected before the EP study and separately from the body surface recordings. An ECG-gated (70% R-R interval) contrast-enhanced cardiac axial CT scan from the level of the great vessel to the diaphragm was performed to obtain the heart geometry with a resolution of 0.39×0.39×0.75 mm. Another thoracic scan from the collar bone level to lower abdomen with a resolution of 0.78×0.78×5 mm was acquired to get a complete torso geometry. These two sets of CT images were registered based on important cardiac anatomical landmarks, such as the apex and the septum between the two ventricles. The registration errors were minimized and a realistic model including torso, lungs, epicardium, and ventricles was built from the CT images by using a commercial software for segmentation and registration.

A total of 100 PVC beats (10 for each patient) were analyzed. Each patient had 10 3D ARI maps calculated from 10 PVC beats in BSPM, which were compared with the patient's one CARTO ARI map to evaluate the performance of ARI imaging. A good correlation was found with an average correlation coefficient (CC) of 0.86±0.05 and an average relative error (RE) of 0.06±0.03. The average localization error between the longest ARI sites in two maps (LE-map) is 4.5±2.3 mm. Based on the activation time maps of activation imaging results, PVC originated from right ventricular outflow tract (RVOT) in 9 out of 10 patients, 7 in the right ventricle free wall side and 2 in the septum side. One patient (patient 2) who was diagnosed as having right bundle branch block (RBBB) had the ectopic beats initiated from left anterior fascicle in the left ventricle. These are in accordance with the clinical ablation locations. From 3D ARI maps of ARI imaging results, the site with the longest ARI was at the same location as the clinical ablation location, with only one exception that for the patient with RBBB, the longest ARI site was in the left posterior fascicle in the left ventricle.

Data for subjects in the present example are reflected in Tables 1 and 2. Table 1 contains modeling and correlation analysis details. Table 2 contains statistics of ablation location and data analysis.

TABLE 1

| | | | | | | | | | | Average |
|---|---|---|---|---|---|---|---|---|---|---|
| | | | | Mean | | | CARTO | | Unipolar | Registration |
| | BSPM | Body | Body | Triangle | Ventricle | Endocardial | Endocardial | Correlation | Recording | Error between |
| | Recorded | Surface | Surface | Edge Length | Volume | Surface | Surface | Analysis | Site | CARTO and CT |
| Pt | (minutes) | Nodes | Triangles | (mm) | Points | Points | Points | Points | (No.) | (mm) |
| 1 | 10 | 3445 | 6886 | 10.76 | 3904 | 408 | 1486 | 408 | 41 | 3.24 |
| 2 | 24 | 1869 | 3734 | 15.62 | 6533 | 1085 | 1573 | 1085 | 34 | 5.45 |
| 3 | 12 | 4698 | 9392 | 10.7 | 6398 | 1050 | 1378 | 1378 | 13 | 6.13 |
| 4 | 14 | 1061 | 2118 | 20.52 | 3552 | 883 | 3195 | 883 | 40 | 3.93 |
| 5 | 21 | 4565 | 9126 | 10.8 | 3649 | 682 | 909 | 909 | 18 | 5.37 |
| 6 | 13 | 1320 | 2636 | 20.54 | 7513 | 1231 | 864 | 864 | 52 | 5.58 |
| 7 | 14 | 2396 | 4788 | 15.74 | 5800 | 1105 | 754 | 754 | 82 | 5.91 |
| 8 | 21 | 2066 | 4128 | 15.73 | 6764 | 1263 | 631 | 631 | 23 | 6.8 |
| 9 | 24 | 1667 | 3330 | 15.7 | 3603 | 815 | 546 | 546 | 11 | 4.36 |
| 10 | 17 | 2034 | 4064 | 15.76 | 3946 | 808 | 1431 | 1431 | 22 | 4.55 |

TABLE 2

| | | | Recorded | Imaged | PVC | | | | | | | |
|---|---|---|---|---|---|---|---|---|---|---|---|---|
| | | Ablation | Longest | Longest | beat | QRS | ST | T-wave | | | LE-map | Dis-actiari |
| Pt | Diagnosis | Location | ARI site | ARI site | (ms) | (ms) | (ms) | (ms) | CC | RE | (mm) | (mm) |
| 1 | PVC | RVOT | RVOT | RVOT | 362 | 101 | 130 | 131 | 0.83 ± 0.01 | 0.03 ± 0.01 | 4.1 ± 0.6 | 4.1 ± 0.9 |
| | | free wall | free wall | free wall | | | | | | | | |
| 2 | PVC, RBBB | Left | Left | Left | 379 | 88 | 125 | 166 | 0.86 ± 0.04 | 0.05 ± 0.01 | 7.7 ± 2.0 | N/A |
| | | Anterior | Posterior | Posterior | | | | | | | | |
| | | Fascicle | Fascicle | Fascicle | | | | | | | | |
| 3 | PVC, | RVOT | RVOT | RVOT | 362 | 113 | 78 | 171 | 0.84 ± 0.02 | 0.04 ± 0.02 | 3.4 ± 2.3 | 4.1 ± 2.5 |
| | Nonsustained | free wall | free wall | free wall | | | | | | | | |
| | VT | | | | | | | | | | | |
| 4 | PVC, | RVOT | RVOT | RVOT | 416 | 113 | 136 | 167 | 0.81 ± 0.02 | 0.08 ± 0.02 | 3.4 ± 1.1 | 5.0 ± 0.5 |
| | Nonsustained | free wall | free wall | free wall | | | | | | | | |
| | AT | | | | | | | | | | | |
| 5 | PVC | RVOT | RVOT | RVOT | 387 | 119 | 106 | 162 | 0.85 ± 0.01 | 0.11 ± 0.01 | 2.6 ± 0.1 | 3.7 ± 0.2 |
| | | free wall | free wall | free wall | | | | | | | | |
| 6 | PVC, | RVOT | RVOT | RVOT | 394 | 126 | 89 | 179 | 0.84 ± 0.01 | 0.08 ± 0.01 | 5.9 ± 1.0 | 4.8 ± 0.8 |
| | Nonsustained | free wall | free wall | free wall | | | | | | | | |
| | VT | | | | | | | | | | | |
| 7 | PVC | RVOT | RVOT | RVOT | 411 | 111 | 144 | 156 | 0.94 ± 0.01 | 0.06 ± 0.01 | 3.6 ± 0.3 | 3.2 ± 1.0 |
| | | free wall | free wall | free wall | | | | | | | | |
| 8 | PVC | RVOT | RVOT | RVOT | 326 | 113 | 58 | 155 | 0.91 ± 0.04 | 0.05 ± 0.01 | 5.1 ± 1.7 | 3.1 ± 1.1 |
| | | septum | septum | septum | | | | | | | | |
| 9 | PVC | RVOT | RVOT | RVOT | 356 | 92 | 110 | 154 | 0.81 ± 0.06 | 0.01 ± 0.01 | 1.3 ± 0.1 | 4.8 ± 0.7 |
| | | free wall | free wall | free wall | | | | | | | | |
| 10 | PVC | RVOT | RVOT | RVOT | 350 | 97 | 84 | 169 | 0.86 ± 0.03 | 0.06 ± 0.04 | 7.1 ± 2.0 | 3.5 ± 1.3 |
| | | septum | septum | septum | | | | | | | | |
| Av | N/A | N/A | N/A | N/A | 374 | 107 | 106 | 161 | 0.86 ± 0.05 | 0.06 ± 0.03 | 4.5 ± 2.3 | 4.0 ± 1.3 |

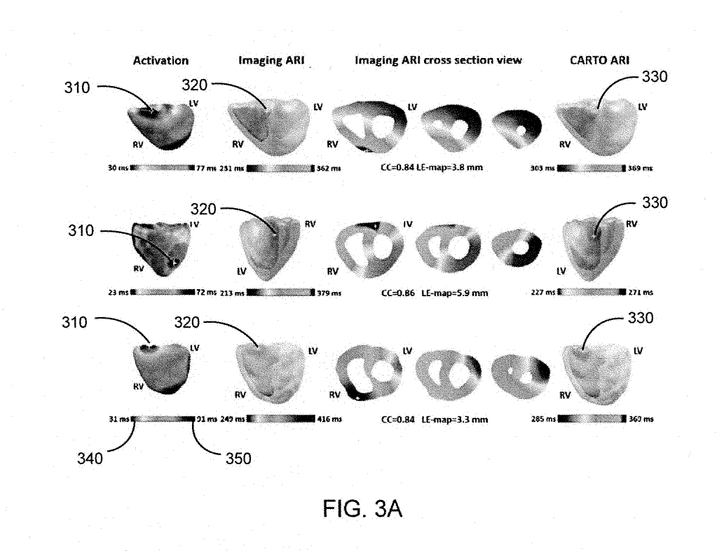

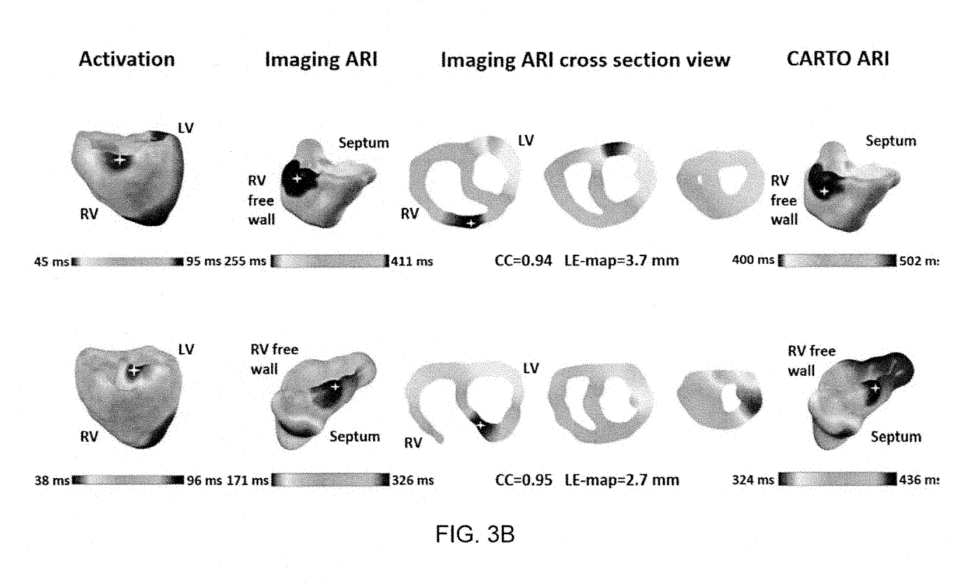

Referring to FIG. 3A, example ARI maps for 3 of the subjects from the example mentioned above are presented with recording sites covering the full cardiac chamber. In some configurations, the maps may be colored to make user interpretation easier. The first column depicts the imaged activation time maps, in which white stars 310 represent the earliest activated sites. The second column refers to the imaged endocardial ARI maps and the fourth column the interpolated CARTO ARI maps. In both columns, the longest ARI sites are labeled stars 320 and 330 respectively. In between, there are three cross section views of imaged 3D ARI maps. All the maps include ranges, with 340 representing the minimum activation time and 350 the maximum activation time. The time scales of imaging ARI maps may be scaled based on the body surface recordings and the time scales of CARTO ARI maps may be determined by the ARI values of recorded sites. The PVC beats analyzed may be marked in Lead II electrograms (not shown). The time segment between select PVC beats in the electrograms may be used for activation imaging and later time points may be the input for recovery imaging. For the top and bottom plots shown in FIG. 3A, the earliest site in activation and the longest ARI sites in both ARI maps are in the same region, namely RVOT right ventricle free wall side. For the middle plot, activation started at left anterior fascicle while the longest ARI site located at left posterior fascicle.

Referring to FIG. 3B, results for two patients in the present example is shown with small CARTO-recorded endocardial surfaces. Because of the small recording area, imaged ARI maps were projected to CARTO-recorded endocardial surfaces and then were compared with interpolated ARI maps quantitatively. The figure format is the same as that in FIG. 3A.

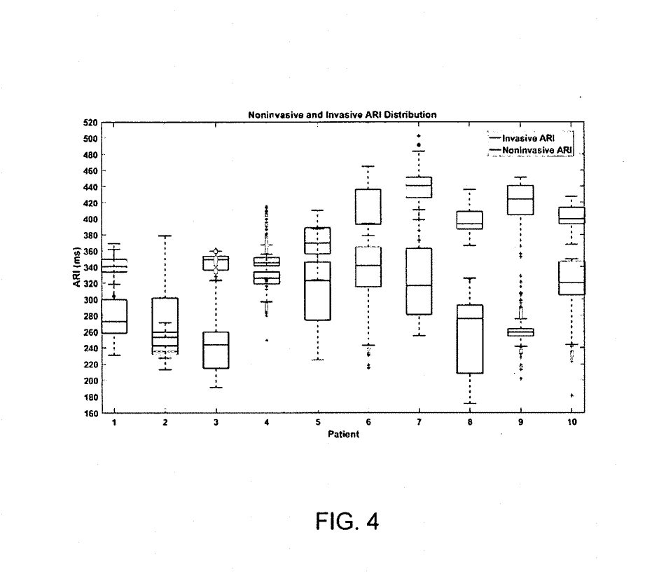

Referring to FIG. 4, an example boxplot is shown that may be used for comparing ARI distributions determined through a invasive, direct-measurement procedure such as CARTO ARI distributions, which may be derived from unipolar electrograms, to noninvasive/imaging-based ARI distributions. Invasive ARI distributions are shown in the upper boxplots and noninvasive ARI distributions are plotted in lower boxplots. The median is the middle line in the box. This form of display may be provided to a user as a way of verifying an ARI map for a particular procedure.

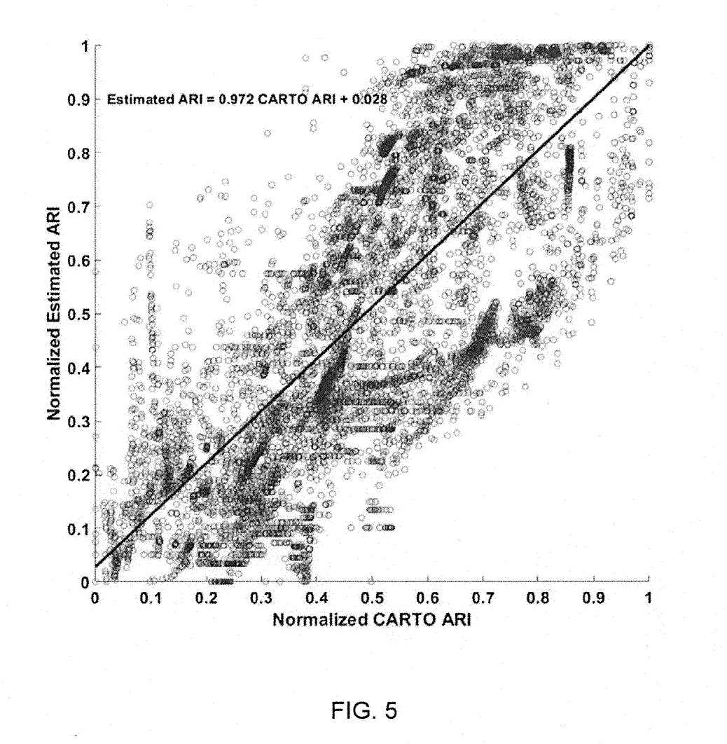

Referring to FIG. 5, an example scatter plot is shown between invasive CARTO ARI and noninvasive imaging ARI. The values are normalized between 0 and 1 in order to plot for all patients. Linear regression may be done between the two ARI data sets. A scatter plot such as that depicted here may be generated by the comparison process depicted in FIG. 1B.

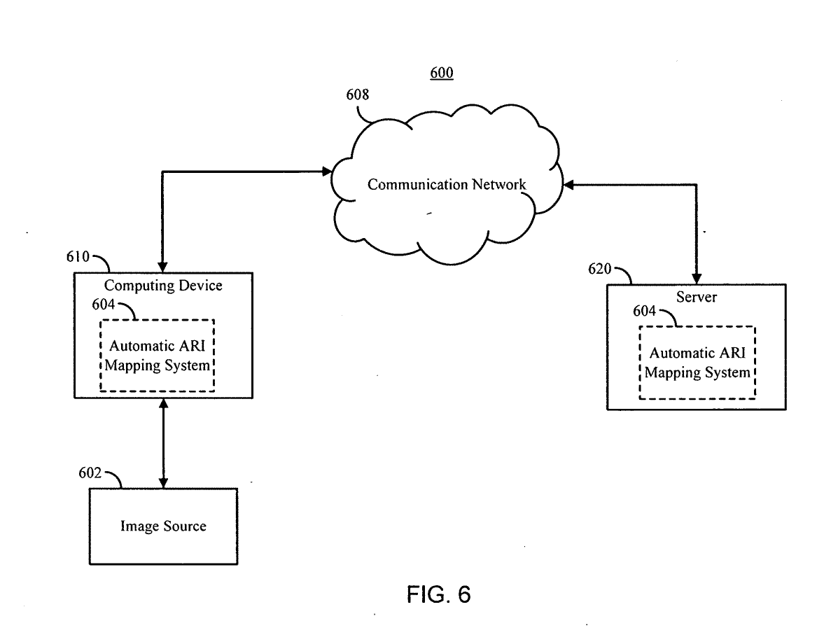

Referring to FIG. 6, an example 600 of a system for automatically generating ARI maps using image data in accordance with some configurations of the disclosed subject matter. A computing device 610 can receive multiple types of image data from image source 602. In some configurations, image source 602 may be a CT system. In some embodiments, computing device 610 can execute at least a portion of an automatic ARI mapping system 604 to automatically determine whether a patient condition, such as a PVC, is present in images of a subject's heart.

Additionally or alternatively, in some embodiments, computing device 610 can communicate information about image data received from image source 602 to a server 620 over a communication network 608, which can execute at least a portion of automatic ARI mapping system 604. In such embodiments, server 620 can return information to computing device 610 (and/or any other suitable computing device) indicative of an output of automatic ARI mapping system 604 to determine whether a clinical condition is present or absent.

In some embodiments, computing device 610 and/or server 620 can be any suitable computing device or combination of devices, such as a desktop computer, a laptop computer, a smartphone, a tablet computer, a wearable computer, a server computer, a virtual machine being executed by a physical computing device, etc. In some embodiments, automatic ARI mapping system 604 can extract features from labeled (e.g., labeled as including diseased or normal) CT image data, respectively, using a convolutional neural network (CNN) trained as a general image classifier.

In some embodiments, image source 602 can be any suitable source of image data, such as a CT machine, another computing device (e.g., a server storing CT image data), etc. In some embodiments, image source 602 can be local to computing device 610. For example, image source 602 can be incorporated with computing device 610 (e.g., computing device 610 can be configured as part of a device for capturing and/or storing images). As another example, image source 602 can be connected to computing device 610 by a cable, a direct wireless link, etc. Additionally or alternatively, in some embodiments, image source 602 can be located locally and/or remotely from computing device 610, and can communicate image data to computing device 610 (and/or server 620) via a communication network (e.g., communication network 608).

In some embodiments, communication network 608 can be any suitable communication network or combination of communication networks. For example, communication network 608 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, communication network 608 can be a local area network, a wide area network, a public network (e.g., the Internet), a private or semi-private network (e.g., a corporate or university intranet), any other suitable type of network, or any suitable combination of networks. Communications links shown in FIG. 6 can each be any suitable communications link or combination of communications links, such as wired links, fiber optic links, Wi-Fi links, Bluetooth links, cellular links, etc.

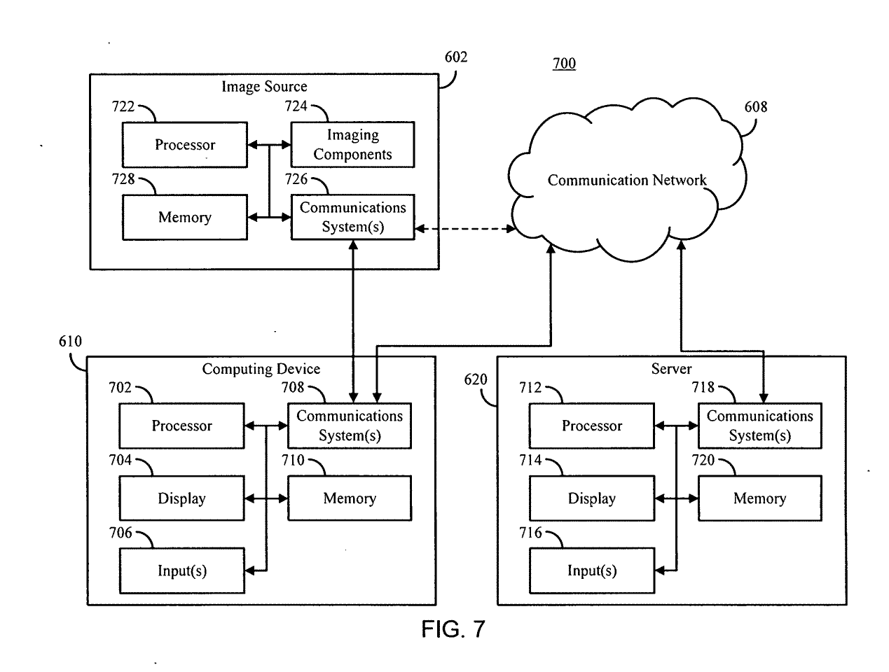

FIG. 7 shows an example 700 of hardware that can be used to implement image source 602, computing device 610, and/or server 620 in accordance with some embodiments of the disclosed subject matter. As shown in FIG. 7, in some embodiments, computing device 610 can include a processor 702, a display 704, one or more inputs 706, one or more communication systems 708, and/or memory 710. In some embodiments, processor 702 can be any suitable hardware processor or combination of processors, such as a central processing unit (CPU), a graphics processing unit (GPU), etc. In some embodiments, display 704 can include any suitable display devices, such as a computer monitor, a touchscreen, a television, etc. In some embodiments, inputs 706 can include any suitable input devices and/or sensors that can be used to receive user input, such as a keyboard, a mouse, a touchscreen, a microphone, etc.

In some embodiments, communications systems 708 can include any suitable hardware, firmware, and/or software for communicating information over communication network 608 and/or any other suitable communication networks. For example, communications systems 708 can include one or more transceivers, one or more communication chips and/or chip sets, etc. In a more particular example, communications systems 708 can include hardware, firmware and/or software that can be used to establish a Wi-Fi connection, a Bluetooth connection, a cellular connection, an Ethernet connection, etc.

In some embodiments, memory 710 can include any suitable storage device or devices that can be used to store instructions, values, etc., that can be used, for example, by processor 702 to present content using display 704, to communicate with server 620 via communications system(s) 708, etc. Memory 710 can include any suitable volatile memory, non-volatile memory, storage, or any suitable combination thereof. For example, memory 710 can include RAM, ROM, EEPROM, one or more flash drives, one or more hard disks, one or more solid state drives, one or more optical drives, etc. In some embodiments, memory 710 can have encoded thereon a computer program for controlling operation of computing device 610. In such embodiments, processor 702 can execute at least a portion of the computer program to present content (e.g., CT images, user interfaces, graphics, tables, etc.), receive content from server 620, transmit information to server 620, etc.

In some embodiments, server 620 can include a processor 712, a display 714, one or more inputs 716, one or more communications systems 718, and/or memory 720. In some embodiments, processor 712 can be any suitable hardware processor or combination of processors, such as a CPU, a GPU, etc. In some embodiments, display 714 can include any suitable display devices, such as a computer monitor, a touchscreen, a television, etc. In some embodiments, inputs 716 can include any suitable input devices and/or sensors that can be used to receive user input, such as a keyboard, a mouse, a touchscreen, a microphone, etc.

In some embodiments, communications systems 718 can include any suitable hardware, firmware, and/or software for communicating information over communication network 608 and/or any other suitable communication networks. For example, communications systems 718 can include one or more transceivers, one or more communication chips and/or chip sets, etc. In a more particular example, communications systems 718 can include hardware, firmware and/or software that can be used to establish a Wi-Fi connection, a Bluetooth connection, a cellular connection, an Ethernet connection, etc.

In some embodiments, memory 720 can include any suitable storage device or devices that can be used to store instructions, values, etc., that can be used, for example, by processor 712 to present content using display 714, to communicate with one or more computing devices 610, etc. Memory 720 can include any suitable volatile memory, non-volatile memory, storage, or any suitable combination thereof. For example, memory 720 can include RAM, ROM, EEPROM, one or more flash drives, one or more hard disks, one or more solid state drives, one or more optical drives, etc. In some embodiments, memory 720 can have encoded thereon a server program for controlling operation of server 620. In such embodiments, processor 712 can execute at least a portion of the server program to transmit information and/or content (e.g., CT data, results of automatic ARI mapping, a user interface, etc.) to one or more computing devices 610, receive information and/or content from one or more computing devices 610, receive instructions from one or more devices (e.g., a personal computer, a laptop computer, a tablet computer, a smartphone, etc.), etc.

In some embodiments, image source 602 can include a processor 722, imaging components 724, one or more communications systems 726, and/or memory 728. In some embodiments, processor 722 can be any suitable hardware processor or combination of processors, such as a CPU, a GPU, etc. In some embodiments, imaging components 724 can be any suitable components to generate image data. An example of an imaging machine that can be used to implement image source 602 can include a conventional CT scanner and the like.

Note that, although not shown, image source 602 can include any suitable inputs and/or outputs. For example, image source 602 can include input devices and/or sensors that can be used to receive user input, such as a keyboard, a mouse, a touchscreen, a microphone, a trackpad, a trackball, hardware buttons, software buttons, etc. As another example, image source 602 can include any suitable display devices, such as a computer monitor, a touchscreen, a television, etc., one or more speakers, etc.

In some embodiments, communications systems 726 can include any suitable hardware, firmware, and/or software for communicating information to computing device 610 (and, in some embodiments, over communication network 608 and/or any other suitable communication networks). For example, communications systems 726 can include one or more transceivers, one or more communication chips and/or chip sets, etc. In a more particular example, communications systems 726 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, etc.

In some embodiments, memory 728 can include any suitable storage device or devices that can be used to store instructions, values, image data, etc., that can be used, for example, by processor 722 to: control imaging components 724, and/or receive image data from imaging components 724; generate images; present content (e.g., CT images, a user interface, etc.) using a display; communicate with one or more computing devices 610; etc. Memory 728 can include any suitable volatile memory, non-volatile memory, storage, or any suitable combination thereof. For example, memory 728 can include RAM, ROM, EEPROM, one or more flash drives, one or more hard disks, one or more solid state drives, one or more optical drives, etc. In some embodiments, memory 728 can have encoded thereon a program for controlling operation of image source 602. In such embodiments, processor 722 can execute at least a portion of the program to generate images, transmit information and/or content (e.g., CT image data) to one or more computing devices 610, receive information and/or content from one or more computing devices 610, receive instructions from one or more devices (e.g., a personal computer, a laptop computer, a tablet computer, a smartphone, etc.), etc.

During ectopic activities such as in PVC or pacing, recovery time is mainly determined by the activation sequence since activation time differences are much larger than local recovery (action potential duration). This is supported by previous findings in which activation time was inversely correlated with ARI during premature stimulation in patients with normal ventricles. So, detecting the longest ARI sites are close to the origins of PVCs in the majority of patients with normal ventricles.

In some configurations, abnormal heart structure may be taken into consideration when interpreting ARI results. Normally, activation is conducted through both bundle branches of the conduction system. However, during RBBB, with the right bundle branch blocked, the right ventricle is activated by the impulses travelled through myocardium from the left ventricle instead of being activated through the bundle of His-Purkinje fibers and the left ventricle is still normally activated by the left bundle branch. Anatomically, the left bundle branch divides into the left anterior fascicles and the left posterior fascicles. Such a situation may result when PVCs are initiated by the left anterior fascicles. ARI imaging results for these types of situations may need to also account for abnormal heart structure in order to ensure that the longest ARI site is close to the earliest activated site.

3D ARI imaging can be potentially applied to any condition that involves recovery abnormalities in the ventricles, including complicated cases. This may include but is not limited to: bundle branch block, early repolarization, cardiac memory, Wolff-Parkinson-White syndrome and ischemia. In the application of the present disclosure to these cases, local recovery gradient may be added into the analysis. Also, to reflect the dynamic changes of recovery and to validate 3D ARI imaging, a simultaneous recording of BSPM and clinical recordings (contact or noncontact unipolar electrograms) may be performed.

The present disclosure has described one or more preferred embodiments, and it should be appreciated that many equivalents, alternatives, variations, and modifications, aside from those expressly stated, are possible and within the scope of the invention.

What is claimed is:

1. A method for cardiac activation—repolarization imaging comprising:
acquiring image data comprising a heart of a subject;
generating a three-dimensional representation of the subject based on the image data and a location of at least one sensor associated with the subject;
acquiring electrophysiological data using the at least one sensor;
reconstructing an activation recovery interval image of the heart of the subject based on the electrophysiological data and the three-dimensional representation of the subject; and
providing the activation recovery interval image of the heart to a user.

2. The method of claim 1, wherein reconstructing the activation recovery interval image further comprises:
reconstructing activation recovery interval distributions based on a time course of current density within the heart of the subject.

3. The method of claim 2, wherein a ventricular myocardium of the heart of the subject is modeled as a plurality of grid points, and wherein the current density at each of the plurality of grid points is determined based on the electrophysiological data.

4. The method of claim 1, wherein reconstructing the activation recovery interval image further comprises:
reconstructing the activation recovery interval image based on a macroscopic continuum model comprising an intracellular domain and an extracellular domain,
wherein the intracellular domain and the extracellular domain are connected by a plurality of transmembrane currents.

5. The method of claim 1, wherein the electrophysiological data comprises body surface potentials measured by the at least one sensor.

6. The method of claim 1, wherein the body surface potentials comprise ECG data of the subject.

7. The method of claim 1, wherein acquiring image data further comprises:
performing a computed tomography (CT) scan of the subject.

8. The method of claim 1, wherein generating the three-dimensional representation of the subject further comprises:
generating the three-dimensional representation of at least one of a torso, lungs, or an epicardium of the subject.

9. The method of claim 1, wherein providing the activation recovery interval image of the heart to the user further comprises:
displaying the activation recovery interval image,
wherein the activation recovery interval image comprises a map indicating an earliest activated site.

10. The method of claim 1, further comprising:
comparing the reconstructed activation recovery interval image to an invasive measurement of the activation recovery interval.

11. A system for cardiac activation—repolarization imaging comprising:
an imaging system for acquiring image data of a heart of a subject;
at least one sensor associated with the subject for acquiring electrophysiological data; and
a processor to:
generate a three-dimensional representation of the subject based on the image data and a location of the at least one sensor,
reconstruct an activation recovery interval image of the heart of the subject based on the electrophysiological data and the three-dimensional representation of the subject, and
provide the activation recovery interval image of the heart to a user.

12. The system of claim 11, wherein the processor, when reconstructing the activation recovery interval image, is further to:
reconstruct activation recovery interval distributions based on a time course of current density within the heart of the subject.

13. The system of claim 12, wherein a ventricular myocardium of the heart of the subject is modeled as a plurality of grid points, and wherein the current density at each of the plurality of grid points is determined based on the electrophysiological data.

14. The system of claim 11, wherein the processor, when reconstructing the activation recovery interval image, is further to:
reconstruct the activation recovery interval image based on a macroscopic continuum model comprising an intracellular domain and an extracellular domain,
wherein the intracellular domain and the extracellular domain are connected by a plurality of transmembrane currents.

15. The system of claim 11, wherein the electrophysiological data comprises body surface potentials measured by the at least one sensor.

16. The system of claim 11, wherein the body surface potentials comprise ECG data of the subject.

17. The system of claim 11, wherein the processor, when acquiring image data, is further to:
perform a computed tomography (CT) scan of the subject.

18. The system of claim 11, wherein the processor, when generating the three-dimensional representation of the subject, is further to:
generate the three-dimensional representation of at least one of a torso, lungs, or an epicardium of the subject.

19. The system of claim 11, wherein the processor, when providing the activation recovery interval image of the heart to the user, is further to:
display the activation recovery interval image,
wherein the activation recovery interval image comprises a map indicating an earliest activated site.

20. The system of claim 11, wherein the processor is further to:
compare the reconstructed activation recovery interval image to an invasive measurement of the activation recovery interval.
